(19) United States
(12) Patent Application Publication
(10) Pub. No.: US 2017/0348280 A1
(43) Pub. Date: Dec. 7, 2017

(54) COUNTERACTING THE INHIBITION OF COLLAGEN SYNTHESIS BY SELECT CALCIUM AND SODIUM CHANNEL BLOCKERS USING ASCORBIC ACID AND ASCORBYL PALMITATE

(71) Applicants: MATTHIAS W RATH, APTOS, CA (US); Aleksandra Niedzwiecki, APTOS, CA (US); Vadim O. Ivanov, CASTRO VALLEY, CA (US); Svetlana V. Ivanova, CASTRO VALLEY, CA (US)

(72) Inventors: MATTHIAS W RATH, APTOS, CA (US); Aleksandra Niedzwiecki, APTOS, CA (US); Vadim O. Ivanov, CASTRO VALLEY, CA (US); Svetlana V. Ivanova, CASTRO VALLEY, CA (US)

(73) Assignee: MATTHIAS W RATH, APTOS, CA (US)

(21) Appl. No.: 15/174,352

(22) Filed: Jun. 6, 2016

(51) Int. Cl. A61K 31/375

(52) U.S. Cl. CPC A61K 31/375

(57) ABSTRACT

A therapeutic use of ascorbyl palmitate, ascorbic acid and/or derivatives thereof to reverse or counter the adverse effects of channel blockers in the medical management of cardiovascular disease in a subject is presented. Effects of select Na- and Ca-channel blockers on collagen synthesis and deposition were evaluated in cultured human dermal fibroblasts and aortic smooth muscle cells by immunoassay. Ca and Na-channel blockers inhibited the synthesis of collagen type I and collagen type IV, the basic molecules of vascular wall stability. The inhibitory effects of the calcium and sodium channel blockers on collagen synthesis was reversed by introducing ascorbic acid and/or ascorbyl palmitate. It can be concluded that calcium and sodium channel blockers have a direct inhibitory effect on collagen synthesis, favoring the instability of the vascular wall and the progression of cardiovascular disease, an effect that is mitigated by treatment with, ascorbyl palmitate, ascorbic acid and or derivatives thereof.

### FIELD OF THE INVENTION

The subject matter disclosed in this application relates generally to the field of new therapeutic uses of ascorbic acid and ascorbyl palmitate to counteract the collagen synthesis inhibition by calcium and sodium channel blockers in human.

### BACKGROUND OF THE INVENTION

Heart failure is a common cardiovascular condition where the heart cannot circulate enough blood and oxygen to meet the needs of other body organs. Heart failure caused by heart disease is a serious condition and there is currently no cure. Once diagnosed, medicines are needed for the rest of the person's life. Heart disease is the number one cause of death in the United States, surpassing even cancer, and accounting for more than 25 percent of all deaths. Heart disease is the leading cause of death for both men and women.

One form of cardiovascular disease, arrhythmia, is associated with very high levels of morbidity and mortality. Sudden arrhythmic death claims more than 300,000 lives each year. Arrhythmia is defined as abnormal beating of the heart. The heart beat is a complex process of contraction and expansion and is controlled by electrical impulses, which are, in turn, regulated by the flow of specific ions (potassium ion—K⁺, sodium ion—Na⁺ and calcium ion—Ca²⁺) across cellular membranes. Integral membrane proteins, or channels, act as gates, controlling the flow of ions in and out of cells. Sodium (Na), calcium (Ca) and potassium (K) channels play pivotal roles in generating cardiac action potential, which triggers contraction. Ion channel dysfunction resulting from genetic mutation and other causes is a primary cause of arrhythmia.

Arrhythmias, such as ventricular tachycardia and ventricular fibrillation can lead to sudden cardiac arrest. A variety of antiarrhythmic agents or drugs, including channel blockers are employed to manage cardiac pathologies. The ultimate goal of antiarrhythmic drug therapy is to restore normal rhythm and conduction. When it is not possible to revert to normal sinus rhythm, drugs may be used to prevent more serious and possibly lethal arrhythmias from occurring. All antiarrhythmic drugs directly or indirectly alter membrane ion conductance, which in turn alters the physical characteristics of cardiac action potentials. For example, some drugs which function as sodium channel blockers are used to block fast sodium channels. Sodium-channel blockers comprise the Class I (membrane stabilizing agents) antiarrhythmic compounds according to the Vaughan-Williams classification scheme [Sampson K J et.al. 2011,]. These drugs bind to and block the fast sodium channels that are responsible for the rapid depolarization (phase 0) of fast-response cardiac action potentials. The principal effect of blocking sodium channels is that it reduces the velocity of action potential transmission within the heart. This type of medication can serve as an important mechanism for suppressing tachycardias that are caused by abnormal conduction [3]. Na blockers, which include such drugs as quinidine and procainamide, are used for treating supraventricular tachycardia, ventricular tachycardia, symptomatic ventricular premature beats, and prevention of ventricular fibrillation. Side effects of quinidine include blurred vision, tinnitus, headache, psychosis, cramping and nausea, and enhancement of digitalis toxicity [3]. Capsaicin is considered a natural Na channel blocker. Most local anaesthetics used clinically are relatively hydrophobic molecules that gain access to their blocking site on the sodium channel by diffusing into or through the cell membrane. These anaesthetics block sodium channels and thereby the excitability of all neurons, not just sensory neurons [4].

Another Class of drugs used in the treatment of arrhythmias is calcium-channel blockers (CCBs), which comprise the Class IV antiarrhythmic compounds according to the Vaughan-Williams classification scheme. This class derives its main therapeutic effects by preventing calcium ion influx through the cell membranes. Calcium channel blockers inhibit the movement of calcium through slow calcium channels of cell membranes in the myocardium, cardiac conduction tissues, and vascular smooth muscle [1]. Currently approved calcium channel blockers bind to L-type calcium channels located on the vascular smooth muscle, cardiac myocytes, and cardiac nodal tissue (sinoatrial and atrioventricular nodes) [5]. They are divided into two categories based upon their predominant physiologic effects: dihydropirydins (predominantly vasodilators) such as nifedepine and non-dihydropyridines (reduce vascular permeability affecting cardiac contractility and conduction) such as verapamil and dilitiazem. Dihydropyridines may cause reflex tachycardia, flushing, headache, and ankle swelling. Ditiazem and verapamil depress cardiac conduction and cause bradycardia. Natural Ca′ channel blockers include magnesium, due to magnesium's ability to block calcium from entering muscle and heart cells, but with less powerful effects than other Ca⁺⁺ channel blockers.

Calcium, sodium and potassium channel blockers are widely prescribed medications for a variety of health problems and are most frequently prescribed for cardiac arrhythmias, hypertension, angina pectoris and other disorders. However chronic application of these agents is associated with problematic side effects. For example, nifedipine, a calcium-channel blocker was found to be associated with increased mortality and increased risk for myocardial infarction [6, 7] and breast cancer (JAMA 2013 study).

In addition to the side effects mentioned above by different channel blockers, these drugs can cause arterial wall damage. For example, Roth et al. reported that Ca′ channel blockers such as verapamil, ditiazem and others significantly decreased the constitutive and platelet-derived growth factor-dependent collagen deposition in the extra cellular matrix ECM formed by human vascular smooth muscle cells and fibroblasts [8]. The drugs inhibited the expression of fibrillary collagens type I and III and of basement membrane type IV collagen [8]. Furthermore, Ca⁺⁺ channel blockers specifically increased the proteolytic activity of the 72-kDa types IV collagenase [8], thereby contributing to vascular wall structure destabilization and promoting events facilitating plaques rupture. Collagen is a critical component of vascular walls, cartilage and is an essential component of connective tissue in the body. Ascorbic acid also referred to as ascorbate, regulates collagen synthesis and is essential for the hydroxylation of lysine and proline, a precondition for optimum crosslinking of collagen and elastin and, thus, for maintaining the integrity of the vascular wall. The major pathway of ascorbic acid cellular uptake is dependent on ion-channel dependent transporters.

Ascorbic acid is essential for the production of collagen and other connective tissue components in the body. Most animals synthesize their own ascorbic acid (vitamin C) according to needs. However, about 40 million years ago this ability was lost as a result of a genetic mutation. Therefore, humans need to obtain ascorbic acid from their diet or through nutritional supplements. Ascorbic acid is probably the most effective, efficient and least toxic antioxidant. Ascorbic acid has several physiological functions which include protecting DNA, enzyme, protein and lipids from oxidative damage and thereby preventing aging, coronary heart diseases, cataract formation, degenerative diseases and cancer. It is essential for collagen synthesis, pro-teoglygans and various components of the extra cellular matrix (ECM). As mentioned ascorbic acid is involved in the hydroxylation of lysine and proline for which ascorbic acid functions as a cofactor. Any deficiency of ascorbic results in impaired collagen formation, which leads to tissue weakness and eventually, scurvy [9]. Scurvy is a condition resulting from a complete bodily depletion of ascorbic acid. It is a fatal disease characterized by the slow dissolution of connective tissue throughout the body, including the walls of the blood vessels.

According to some experts in the field including Rath and others [9], the coronary arteries typically suffer the scorbutic (scurvy) effect from the lack of ascorbic acid, which results in impaired collagen synthesis in the body. The coronary arteries begin to deteriorate first because they are subject to the greatest degree of mechanical stress from the pumping action of the beating heart. Without collagen the ground substance (intima) of the blood vessels becomes weak and watery, which in turn allows low-density lipoprotein (LDL) and lipoprotein(a) (Lp(a)) cholesterol to penetrate the tissue to “patch” the blood vessels so that they do not rupture. LDL and lipoprotein(a) deposit in the vascular walls as reinforcing factors, but, at the same time, contribute to a buildup of atherosclerotic plaques and increase the risk of heart attack or stroke. Recent study by Cha et al. [1] confirmed this concept stressing an importance of vascular collagen synthesis in preventing vascular deposition of lipoproteins and development of atherosclerosis.

The wide use of channel blockers, which have to be taken for decades or for life and its negative impact on collagen production, would imply that over time it has a detrimental effect on the vascular system resulting in further cardio vascular disease. In addition it may cause damage to other essential components of the extracellular matrix. There is a need to counteract the calcium and sodium channel blockers and improve the vascular health of human being.

### SUMMARY OF THE INVENTION

The present invention discloses a new use for ascorbic acid and ascorbyl palmitate. In one embodiment of this invention a new method of therapy for the treatment of heart diseases and mitigation of the side effects of widely used pharmaceutical antiarrhythmic drugs is disclosed. Therefore, one embodiment this study was to evaluate effects of various types of channel blockers on intracellular accumulation and cellular functions of ascorbate, specifically in relation to formation and extracellular deposition of major collagen types relevant to vascular function.

This disclosure relates to the potential of employing ascorbate and ascorbyl palmitate to reverse the adverse effects of channel blockers in the medical management of cardiovascular disease. More specifically, the subject matter discloses effects of various types of channel blockers on intracellular accumulation and cellular functions of ascorbate, specifically in relation to formation and extracellular deposition of major collagen types relevant for vascular function.

In one embodiment, group I and group IV antiarrhythmic agents are used and ascorbate treatment is combined to counteract the inhibition of collagen formation in human. Group I antiarrhythmic agents are sodium channel blockers, which block the fast sodium channels, thereby slowing electrical conduction in the heart. Group IV antiarrhythmic agents are calcium channel blockers, which inhibit the calcium channels reducing the movement of calcium ions in the cells during action potentials. Anti arrhythmic agents, which are sodium and channel blockers are used to manage life, threatening arrhythmias, however, chronic application of channel blockers is associated with numerous side effects, including worsening cardiac pathology. One such example is the drug nifedipine, which is a calcium-channel blocker and is found to be associated with increased mortality and increased risk for myocardial infarction.

In one embodiment, A method of countering counteracting an inhibition of collagen synthesis in a subject suffering from cardiovascular disease comprises of administering an effective amount of an ascorbate to the subject as an adjunct treatment with a channel blocker; measuring the effectiveness of the administration of the ascorbate in relation to the level of a housekeeping intracellular protein beta actin; and measuring a level of at least one of collagen I and collagen V in the subject.

This method enables us to monitor, treat and evaluate the effectiveness of the adjunct administration of ascorbate such as ascorbic acid and ascorbyl palmitate to human when they suffer from cardiovascular disease and take calcium or sodium channel blockers as treatment.

In some embodiments of the present invention it is disclosed that all the channel blockers tested demonstrated inhibitory effects on collagen type I deposition to the ECM by fibroblasts, each to a different degree. In other embodiments of the present invention a method is disclosed to significantly increase, by treatment with ascorbic acid the collagen type I ECM deposition, which is reduced by channel blockers. In another embodiment of the present invention it is disclosed that 50 μM nifedipine, a representative of channel blockers tested, significantly reduced ascorbic acid and ascorbyl palmitate-dependent ECM deposition of collagen type I and collagen type IV by cultured aortic smooth muscle cells. In addition in another embodiment of the present invention it is disclosed that 50 μM nifedipine significantly reduced ascorbate-dependent collagen type I and type IV synthesis by cultured aortic smooth muscle cells, assayed by measuring intracellular collagen content.

In another embodiment of the present invention increased intracellular levels of ascorbate under supplementation with elevated doses of ascorbic acid, as well as its lipid soluble derivative ascorbyl palmitate were observed. In another embodiment of the present invention it was observed that nifedipine reduced ascorbic acid intracellular influx in cultured aortic smooth muscle cells, this was observed with 50 μM nifedipine compared to control. Adverse effects of nifedipine were neutralized either by an increased level of cell supplementation with ascorbic acid or by substituting it with ascorbyl palmitate. These studies suggest that channel blockers cause adverse effects by interfering with proper extracellular matrix formation and thereby weakening the arterial wall integrity. In one embodiment of this invention the ascorbate supplementation reversed channel blocker inhibition of ECM components, which are necessary for optimal structural integrity of the arterial wall, signifying the potential of employing ascorbate and ascorbate palmitate in the medical management of cardiovascular disease to reverse the adverse effects of channel blockers.

Finally, the present invention is described further in the detailed description to further illustrate various aspects of the present invention.

### BRIEF DESCRIPTION OF THE FIGURES

Example embodiments are illustrated by way of example and not limitation in the figures of the accompanying drawings, in which like references indicate similar elements and in which:

FIG. 1A illustrates the effects of capsaicin (natural Na⁺ blocker) on collagen type I ECM deposition by cultured human skin fibroblasts. (* indicates the significance of p≦0.005 between ascorbate and ascorbate+ capsaicin ECM collagen 1 deposition)

FIG. 1B illustrates the effects of ditiazem (Ca⁺⁺ blocker) on collagen type I ECM deposition by human skin fibroblasts (* indicates significance of p≦0.0001 between ascorbate vs. ascorbate and ditiazem ECM collagen 1 deposition)

FIG. 1C illustrates the effects of lidocaine (Na⁺ blocker) on collagen type I ECM deposition by cultured human skin fibroblasts. (* indicates the significance of p≦0.0001 between ascorbate and ascorbate+lidocaine ECM collagen 1 deposition; ** indicates significance of p≦0.01 between ECM collagen 1 deposition at increased concentrations of lidocaine compared to control)

FIG. 1D illustrates the effects of nifedipine (Ca⁺⁺ blocker) on collagen type I ECM deposition by cultured human skin fibroblasts. (* indicates the significance of p<0.0001 between ascorbate and ascorbate+nifedipine ECM collagen 1 deposition; ** indicates significance of p≦0.05 between ECM collagen 1 deposition at increased concentrations of nifedipine compared to control)

FIG. 1E illustrates the effects of quinidine (Na⁺ blocker) on collagen type I ECM deposition by cultured human skin fibroblast. (* indicates the significance of p≦0.006 between ascorbate and ascorbate+quinidine ECM collagen 1 deposition)

FIG. 1F illustrates the effects of verapamil (Ca⁺⁺ blocker) on collagen type I ECM deposition by cultured human skin fibroblasts. (* indicates the significance of p≦0.002 between ascorbate and ascorbate+verapamil ECM collagen 1 deposition)

FIG. 2A illustrates the effects of 50 μM nifedipine on ascorbic acid dependent ECM deposition of collagen type I by cultured AoSMC (* indicates the significance of p≦0.02 between ascorbate and ascorbate+nifedipine ECM collagen 1 deposition)

FIG. 2B illustrates the Effects of 50 μM nifedipine on ascorbyl palmitate dependent-ECM deposition of collagen type I by cultured AoSMC. (* indicates the significance of p≦0.045 between ascorbyl palmitate and ascorbyl palmitate+nifedipine ECM collagen 1 deposition)

FIG. 2C illustrates the effects of 50 μM nifedipine on ascorbic acid dependent ECM deposition of collagen type IV by AoSMC. (* indicates the significance of p≦0.02 between ascorbate and ascorbate+nifedipine ECM collagen IV deposition)

FIG. 2D illustrates the effects of 50 μM nifedipine on ascorbyl palmitate dependent ECM deposition of collagen type IV by AoSMC. (* indicates the significance of p≦0.02 between ascorbyl palmitate and ascorbyl palmitate+nifedipine ECM collagen IV deposition)

FIG. 3A illustrates the effects of ascorbic acid on intracellular content of collagen type I in AoSMC in the presence or absence of 50 μM nifedipine. (* indicates the significance of p≦0.002 between ascorbic acid or ascorbyl palmitate alone vs. with nifedipine on intracellular content of collagen type 1)

FIG. 3B illustrates the effects of ascorbic acid on intracellular content of beta-Actin in AoSMC in the presence or absence of 50 μM nifedipine. (* indicates the significance of p≦0.03 between ascorbic acid or ascorbyl palmitate alone vs. with nifedipine on intracellular content of beta actin)

FIG. 3C illustrates the effects of ascorbic acid on intracellular content of collagen type I in AoSMC in the presence or absence of 50 μM nifedipine. Collagen type I content was normalized by beta actin content in corresponding samples. (* indicates the significance of p≦0.003 between ascorbic acid alone vs. with nifedipine on intracellular content of collagen type 1)

FIG. 4 illustrates the effects of nifedipine 50 μM on ascorbate accumulation inside human AoSMC. (* indicates the significance of p≦0.06 between ascorbic acid or ascorbyl palmitate alone vs. with nifedipine on intracellular ascorbate content)

Others features of the present embodiments will be apparent from the accompanying drawings and from the detailed description that follows.

### DETAILED DESCRIPTION

The following embodiments are described for illustrative purposes only with reference to the Figures. Those of skill in the art will appreciate that the following description is exemplary in nature, and that various modifications to the parameters set forth herein could be made without departing from the scope of the present invention. It is intended that the specification and examples be considered as examples only. The various embodiments are not necessarily mutually exclusive, as some embodiments can be combined with one or more other embodiments to form new embodiments.

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this disclosure pertains. The use of the word “a” or “an” when used in conjunction with the term “comprising” in the claims and/or the description may mean “one”, but it is also consistent with the meaning of “one or more”, “at least one”, and “one or more than one”. Similarly, the word “another” may mean at least a second or more. As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unmentioned elements or process steps. As used herein, when referring to numerical values or percentages, the term “about” includes variations due to the methods used to determine the values or percentages, statistical variance and human error. Moreover, each numerical parameter in this application should at least be construed in light of the number of reported significant digits and by applying ordinary rounding techniques. The making and using of the presently preferred embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

Heart disease is the leading cause of death for both men and women. Arrhythmia is a kind of heart disease, wherein there is a problem with the rate or rhythm of the heartbeat. The term “arrhythmia” refers to any change from the normal sequence of electrical impulses. The electrical impulses may happen too fast, too slowly, or erratically thereby causing the heart to beat too fast, too slowly, or erratically. In the case where the heart does not beat properly, it cannot pump blood effectively, which leads to improper functioning of the lungs, brain and all other organs and they may eventually shut down or be damaged. Arrhythmias are an abnormality of the rate or rhythm of the heartbeat, such as atrial fibrillation, atrial flutter, ventricular fibrillation and ventricular tachycardia, which can lead to sudden cardiac arrest. Sudden cardiac arrest is the abrupt loss of heart function in a person who may or may not have diagnosed heart disease. The time and mode of death are unexpected. It occurs instantly or shortly after the symptoms appear.

Antiarrhythmic agents are pharmaceuticals used to combat cardiac arrhythmias and a variety of antiarrhythmic agents are employed to manage cardiac pathologies. Antiarrhythmic agents are often classed into five main groups according to their mechanism of action, using the Vaughan-Williams classification. Many of the agents however, that fall in the five groups, have considerable overlap in their pharmacologic properties.

Ascorbic acid is involved in many physiological functions in living organisms. Its role in the synthesis of collagen in connective tissues is well known. The absence of wound healing and the failure of fractures to repair are classically recognized features of scurvy. These features are attributable to impaired collagen formation due to lack of vitamin C. Dr. Rath [9] proposed that heart disease is an early form of scurvy, a condition that increases the needs for biological repair of weakened arterial walls due to impaired collagen synthesis in the body. Ascorbic acid is involved in the metabolism of several amino acids, leading to the formation of hydroxyproline, hydroxylysine, norepinephrine, serotonin, homogenistic acid, and carnitine. Hydroxyproline and hydroxylysine are components of collagens, the fibrous connective tissue in animals. Collagens are principal components of tendons, ligaments, skin, bone, teeth, cartilage, heart valves, intervertebral disks, cornea, eye lens, and the ground substances between cells. Ascorbic acid is essential for the hydroxylation of lysine and proline, a precondition for optimum crosslinking of collagen and elastin and, thus, for maintaining the integrity of the vascular wall. When collagen is synthesized, proline and lysine are hydroxylated to hydroxyproline and hydroxylysine, which are required for the formation of a stable extracellular matrix and cross-links in the collagen fibers. The subsequent triple helix quaternary state of physiologically effective collagen can only be achieved if the requisite proline and lysine residues have been hydroxylated. A deficiency of ascorbic acid reduces the activity of two mixed-function oxidases, prolylhydroxylase and lysyl hydroxylase, which hydroxylate proline and lysine. Some collagen does form in the absence of ascorbic acid, but the fibers are abnormal, resulting in skin lesions and blood vessel fragility, characteristics of scurvy. The major pathway of ascorbic acid cellular uptake is dependent on ion-channel dependent transporters. Therefore, the objective in this study was to evaluate possible interference of select channel blockers on intracellular accumulation and cellular functions of ascorbate in relation to extracellular matrix (ECM) formation. Effects of select Na⁺ and Ca²⁺ channel blockers on collagen synthesis and deposition were evaluated in cultured human dermal fibroblasts and aortic smooth muscle cells by immunoassay.

### Materials and Methods

All reagents were from Sigma-Aldrich (St. Louis, Mo.) except when indicated differently. Normal human dermal fibroblasts (DF) were supplied by ATCC (Manassas, Va.). Human aortic smooth muscle cells (AoSMC) were purchased from Cambrix (East Rutherford, N.J.). Both cell cultures were maintained in DMEM medium (ATCC) containing antibiotics and 5% fetal bovine serum (FBS, ATCC). All cell cultures were maintained at 37° C. and 5% CO₂ atmosphere. Cell viability was monitored with MTT assay. None of the used experimental conditions resulted in statistically significant cell death (data not shown).

Collagen production by human cultured cells: In the case of these experiments, DF or AoSMC, at 5th to 8th passages, were seeded on collagen type I-covered plastic plates (Becton-Dickinson, collagen I isolated from rat tail tendon) at density of 25,000/cm² and grown to confluence for 5-7 days. For intracellular immunoassay experiments, plain plastic 96-well plates were used. Tested compounds were added to cells at indicated concentrations for 72 h in DMEM supplemented with 2% FBS and cell-produced extracellular matrix was exposed by sequential treatment with 0.5% Triton X100 and 20 mM ammonium sulfate in phosphate buffered saline (PBS, Life Technologies) for 3 min each at room temperature [11]. After four washes with PBS, ECM layers were treated with 1% bovine serum albumin (BSA) in PBS for one hour at room temperature and immediately used in experiments. Alternatively, cell layers were washed three times with PBS and fixed with 3% formaldehyde in PBS at 4° C. for one hour. Fixed cell layers were washed four times with PBS and treated with 1% BSA/PBS for one hour at room temperature.

Collagen and beta-actin immunoassays: Immunoassays were done [12] by sequential incubation with primary monoclonal antibodies specific to human collagen type I or IV or human beta-actin in 1% BSA/PBS for 2 hours followed by 1 hour incubation with secondary goat anti-mouse IgG antibodies labeled with horse radish peroxidase (HRP). Retained peroxidase activity was measured after the last washing cycle (three times with 0.1% BSA/PBS) using TMB peroxidase substrate reagent (Rockland). Optical density was read with plate reader (Molecular Devices) at 450 nm and expressed as a percentage of control cell samples incubated in un-supplemented 1% BSA/DMEM.

Intracellular ascorbic acid assay: AoSMC cultures were seeded and grown to confluent layers in plastic 24-well plates (Becton-Dickinson) and treated with nifedipine and ascorbates for 90 min as described above. Intracellular ascorbic acid was extracted as described by Lane and Lawen [13]. Cells were washed briefly three times with ice-cold PBS on ice and incubated with 300 μl of 0.1% saponin in 1% ethanol in PBS for 10 min on an orbit mixer. Samples were collected to Eppendorf tubes and centrifuged at 13,000×g for 5 min. Supernatants were used in an ascorbic acid assay (Ascorbic Acid Colorimetric Assay Kit II FRASC, BioVision) in accordance with the manufacturer's protocol.

Statistical analysis: Results in figures are means±SD from three or more repetitions from the more representative of at least two independent experiments. Differences between samples were estimated with a two-tailed Student's t-test using MedCalc software (Mariakerke, Belgium) and accepted as significant at p levels less than 0.05.

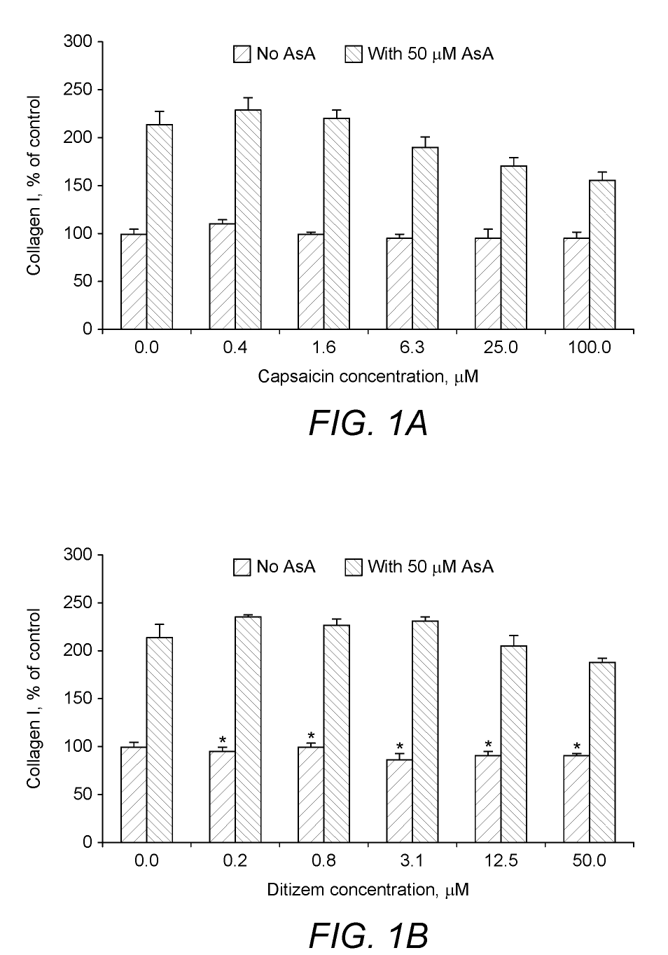

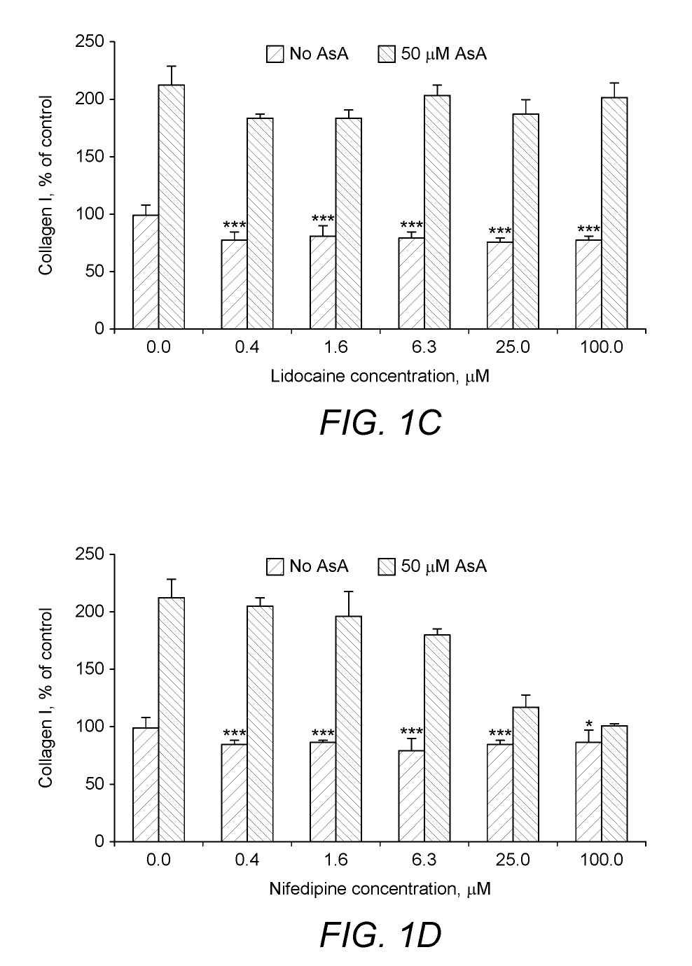

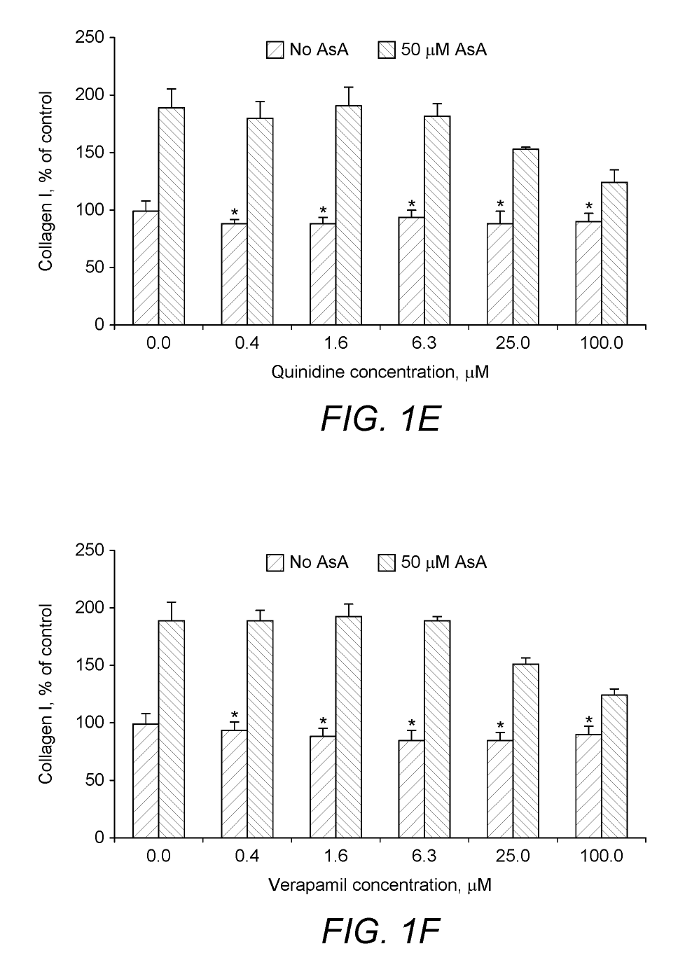

Effect of channel blockers and ascorbate on collagen type I ECM deposition by fibroblasts: The results presented in the FIGS. 1A to 1F show one embodiment of this invention wherein all the channel blockers tested inhibited collagen type I deposition to the ECM in the human skin fibroblasts culture however, each one of them do so to a different degree. In another embodiment of this invention it is disclosed that in the presence of ascorbic acid the ECM deposition of collagen I is significantly increased. Among the tested channel blockers, capsaicin demonstrated slight, insignificant inhibition of collagen type I. However, in the presence of 50 μM of ascorbate the collagen I deposition significantly increased (164%-219%, p≦0.0005) at each concentration of capsaicin compared to the level of collagen I deposited under capsaicin without ascorbate.

In one embodiment of this invention, as presented in FIG. 1B ditiazem showed up to 13.1% inhibition of collagen type I, but this difference did not reach statistical significance. Trend line R²=0.5045. However, in another embodiment of this invention it was shown that in the presence of 50 μM ascorbate, collagen I deposition significantly increased from 206% to 266%, (p≦0.0001) at each concentration of ditiazem compared to the level of collagen I deposited under ditiazem without ascorbate.

In one embodiment of this invention, as presented in FIG. 1C it is shown that in the presence of lidocaine there was significant (19.3%-23.7%, p≦0.01) inhibition of collagen type I, trend line R²=0.4756. In another embodiment of this invention, it is seen that treatment with 50 μM ascorbate resulted in a significant increase of collagen I, from 237% to 258% (p≦0.0001), at each concentration of lidocaine compared to the level of collagen I deposited under lidocaine only.

In one embodiment of this invention, the effects of nifedipine on collagen type I deposition to the ECM in the human skin fibroblasts culture are presented on FIG. 1D. They show a significant (13%-20.3% (p≦0.05) inhibition of collagen type I, (except when 100 μM concentrations were used), trend line R²=0.3628. In another embodiment of this invention it is shown that in the presence of 50 μM ascorbate there was a significant increase of collagen type I deposition to the ECM in the human skin fibroblasts culture (117%-244%, p≦0.0001) at each concentration of nifedipine compared to the level of collagen I deposited under nifedipine without ascorbate. This effect of nifedipine on collagen type I deposition to the ECM in the human skin fibroblasts culture is shown in FIG. 1D.

In one embodiment of this invention it is shown that in the presence of quinidine there was 6% to 12.2% inhibition of collagen type I but the difference did not reach statistical significance, trend line R²=0.2047 as seen in FIG. 1E. In another embodiment of this invention it is seen that by including ascorbate at 50 μM concentration, there was a significant increase of collagen I deposition to the ECM in the human skin fibroblasts culture (137%-217%, p≦0.006) at each concentration of quinidine compared to the level of collagen I deposited under quinidine without ascorbate.

In one embodiment of this invention as shown in FIG. 1F it is observed that in the presence of verapamil there was 5.6% to 15.5% inhibition of collagen type I by this calcium channel blocker, but the difference did not reach statistical significance, trend line R²=0.5383. In another embodiment of this invention it is seen that by including 50 μM ascorbate the collagen type I deposition to the ECM in the human skin fibroblasts culture significantly increased (137%-224%, p≦0.002) at each concentration of verapamil compared to the level of collagen I deposited under verapamil without ascorbate.

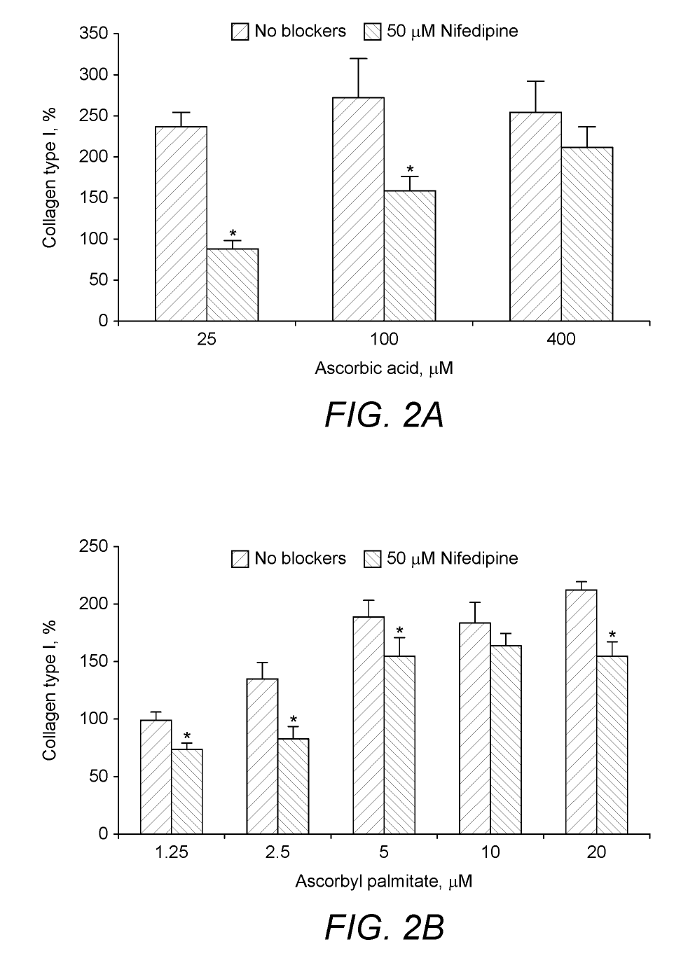

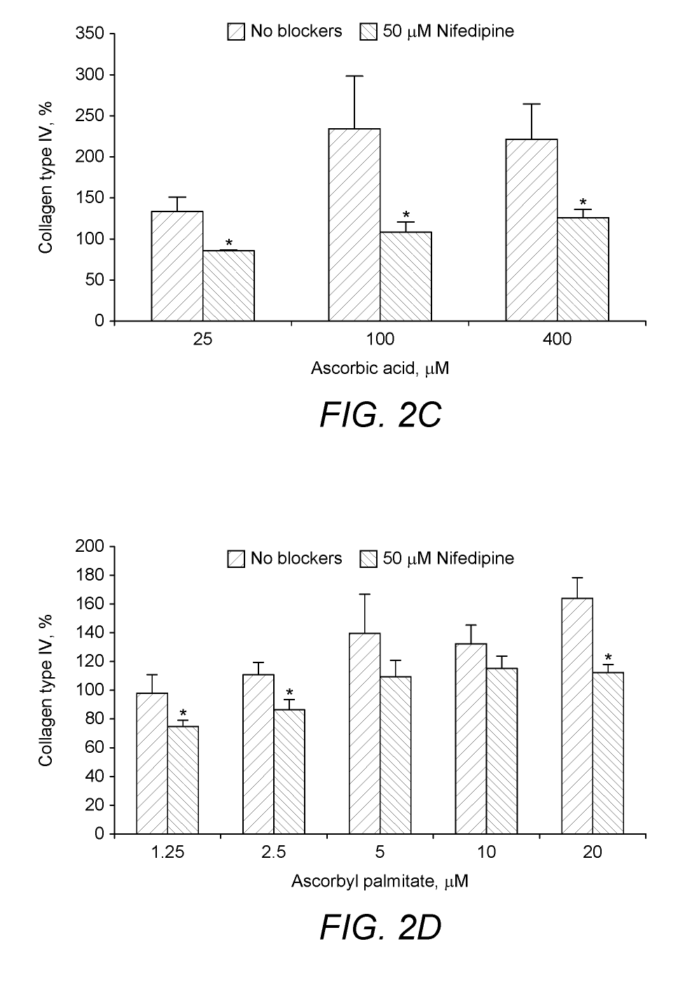

Effect of nifedipine, ascorbic acid and ascorbyl palmitate on collagen types I and IV deposition by cultured human aortic smooth muscle cells (AoSMC): In one embodiment of this invention nifedipine, a representative member of the drugs of the channel blockers class had similar effects on ascorbic acid dependent collagen type I and type IV deposition in AoSMC as it did in human skin fibroblast culture. In another embodiment of this invention comparable results were obtained with verapamil and quinidine (not shown). In one embodiment of this invention as presented in FIG. 2A, 50 μM nifedipine significantly reduced ascorbic acid dependent deposition of collagen type 1 to the ECM in the aortic smooth muscle cells culture: 62.3% (p=0.0001), 41.8% (p=0.019) and 16.5% (p=0.177) at 25, 100 and 400 μM ascorbic acid, respectively. Trend line for ascorbic acid with nifedipine R²=0.995. It is observed that ascorbic acid at 100 and 400 μM increased the ECM deposition by 114% and 176%, respectively, compared to 25 μM ascorbic acid. Trend line R²=0.2512. In another embodiment of this invention it is observed that 50 μM nifedipine significantly reduced ascorbyl palmitate dependent deposition of collagen type 1 to the ECM in the aortic smooth muscle cells culture: 25% (p=0.005), 38% (p=0.006), 27.5% (p=0.045), 11% (p=0.154) and 26.5% (p=0.0025) at 1.25, 2.5, 5, 10 and 20 μM ascorbyl palmitate, respectively, as shown in FIG. 2B. Trend line for ascorbyl palmitate with nifedipine R²=0.7695. It is observed that ascorbyl palmitate administered individually increased ECM deposition by 135% (p=0.013), 189% (p=0.004), 184% (p=0.0013) and 212% (p≦0.0001) at 2.5, 5, 10 and 20 μM, respectively, compared to 1.25 μM ascorbyl palmitate. Trend line R²=0.90.

As presented in FIG. 2C, in the presence of 50 μM nifedipine the ascorbic acid dependent deposition of collagen type IV to the ECM in the aortic smooth muscle cells culture was significantly reduced: 36% (p=0.0029), 54% (p=0.021) and 43% (p=0.02) at 25, 100 and 400 μM ascorbic acid, respectively. Trend line for ascorbic acid with nifedipine R²=0.9982. Ascorbic acid showed increased collagen IV ECM deposition by 175% (p=0.053) and 166% (p=0.0265) at 100 and 400 μM, respectively, compared to 25 μM ascorbic acid, trend line R²=0.652.

FIG. 2D shows the effects of 50 μM nifedipine on ascorbyl palmitate dependent ECM deposition of collagen type IV to the ECM in the aortic smooth muscle cells. The results show a significant decrease in collagen IV to 23.5% (p=0.0459), 22.7% (p=0.0228), 22% (p=0.139), 13.5% (p=0.093) and 31.6% (p=0.0055) at 1.25, 2.5, 5, 10 and 20 μM ascorbyl palmitate, respectively. Trend line for ascorbyl palmitate with nifedipine R²=0.8332. Ascorbyl palmitate alone showed increased collagen IV deposition: 113% (p=0.228), 144% (p=0.068), 138% (p=0.023) and 168% (p=0.004) at 2.5, 5, 10 and 20 μM, respectively, compared to 1.25 μM ascorbyl palmitate, trend line R²=0.8976.

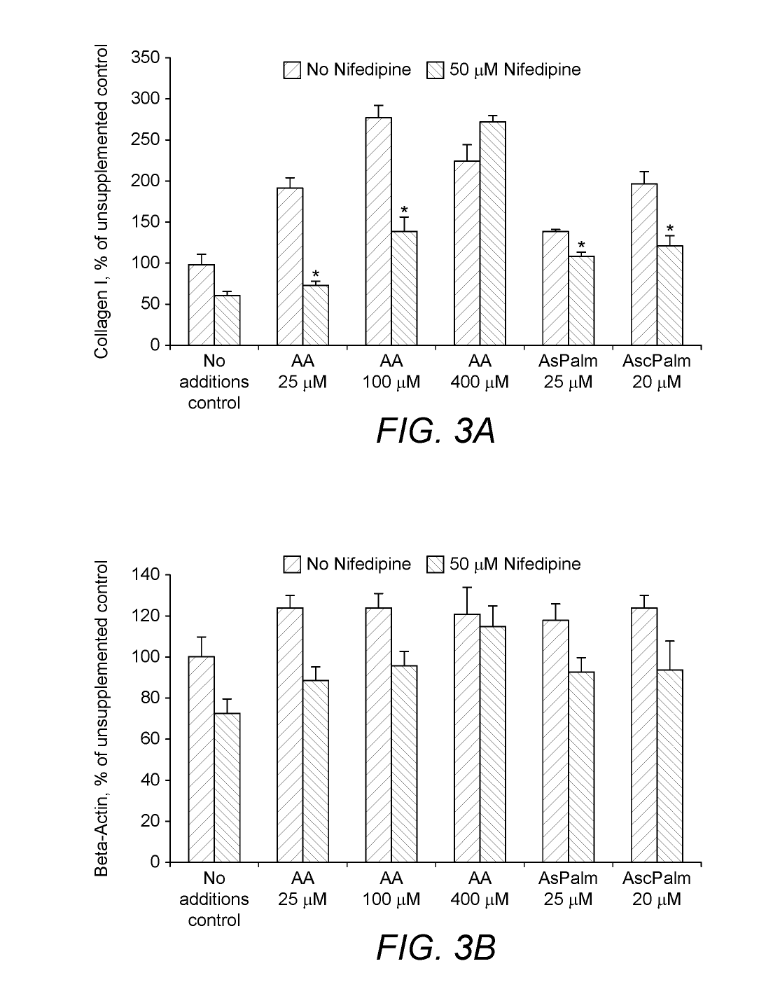

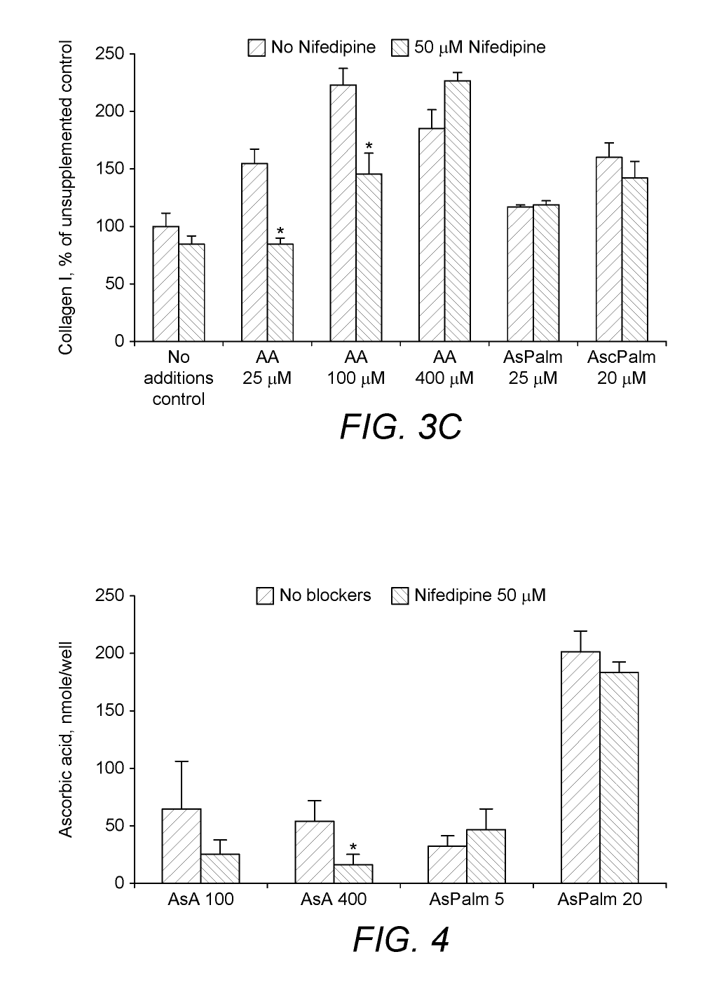

Effect of nifedipine on ascorbate-dependent stimulation of collagen type I synthesis in AoSMC: In these experiments the intracellular collagen content was evaluated in relation to the levels of housekeeping intracellular protein, beta-actin. As shown in FIG. 3A, 50 μM nifedipine significantly reduced ascorbate-dependent intracellular collagen type 1 synthesis by cultured aortic smooth muscle cells, by 61% (p=0.0002) and 49.1% (p=0.0005) at 25 and 100 μM ascorbic acid, respectively. In the presence of 400 μM ascorbic acid and nifedipine the intracellular collagen I increased by 122% (p=0.0195) compared to control. In the presence of ascorbyl palmitate at 5 and 20 nifedipine significantly reduced ascorbate-dependent collagen type 1 synthesis by 20.3% (p=0.0002) and 38% (p=0.002), respectively. Trend line for ascorbic acid with nifedipine R²=0.638 and for ascorbyl palmitate with nifedipine R²=0.9056. Trend line for ascorbic acid effect alone on collagen I intracellular content was R²=0.874 and that for ascorbyl palmitate alone was R²=0.985.

Nifedipine (50 μM) significantly reduced ascorbate-dependent beta-actin intracellular content in cultured aortic smooth muscle cells compared to control: 29%% (p=0.0018), 22.6% (p=0.0061) and 5% (p=0.54) at 25, 100 and 400 μM ascorbic acid, respectively, as shown in FIG. 3B. In the presence of 5 μM and 20 μM of ascorbyl palmitate nifedipine significantly reduced intracellular beta actin content by 22% (p=0.013) and 23.6% (p=0.033), respectively. Trend line for ascorbic acid with nifedipine R²=0.5157 and for ascorbyl palmitate with nifedipine R²=0.8088. Trend line for ascorbic acid effect alone on collagen I intracellular content was R²=0.5157 and that for ascorbyl palmitate alone was R²=0.914.

In the presence of 50 μM nifedipine and ascorbate the intracellular content of collagen type I normalized to beta actin content in aortic smooth muscle cells was reduced compared to the control: 46% (p=0.0005) and 34.4% (p=0.0033) at 25 and 100 μM ascorbic acid, respectively as shown in FIG. 3C. In the presence of 400 μM ascorbic acid nifedipine increased collagen I by 23% (p=0.017) compared to control. However, nifedipine had no significant effect on collagen I content in the presence of ascorbyl palmitate at 5 μM and 20 μM concentrations. Trend line for ascorbic acid with nifedipine R²=0.871 and for ascorbyl palmitate with nifedipine R²=0.9918. Trend line for ascorbic acid effect alone on collagen I intracellular content was R²=0.6396 and that for ascorbyl palmitate alone was R²=0.9442.

Effect of nifedipine on intracellular accumulation of ascorbic acid in AoSMC: Nifedipine (50 μM) had an inhibitory effect on intracellular accumulation of ascorbate in cultured aortic smooth muscle cells compared to the control. As shown in FIG. 4, in the presence of 100 μM ascorbic acid, nifedipine decreased intracellular ascorbate by 61.5% (p=0.161) but this difference did not reach statistical significance. However, at 400 μM ascorbic acid, nifedipine significantly reduced (67.7%, p=0.0337) ascorbate intracellular content. However, nifedipine had no significant effect on intracellular ascorbate levels in the cells incubated with ascorbyl palmitate. It was observed that, supplementation with 20 ascorbyl palmitate resulted in significantly higher (10×, P<0.0001) intracellular levels of ascorbic acid compared to the highest concentrations of ascorbic acid used in experiments, which was 400 Ascorbyl palmitate levels higher than 20 μM were found to be cytotoxic to cultured fibroblasts and smooth muscle cells under the experimental conditions (data not shown).

All channel blockers tested demonstrated inhibitory effects on collagen type I deposition to the ECM by fibroblasts, each to a different degree. Ascorbic acid significantly increased collagen Type I ECM deposition. Nifedipine, a representative member of the channel blockers class, displayed similar effects on ascorbic acid and on ascorbyl palmitate dependent collagen Type I deposition by AoSMC and by human dermal fibroblasts. Furthermore, nifedipine demonstrated significant inhibition of collagen IV ECM deposition.

By determining intracellular collagen content in relation to the levels of housekeeping intracellular protein beta-actin, it was found that nifedipine counteracted ascorbate-dependent stimulation of collagen type I synthesis in AoSMC. Thus, changes in ECM deposition of collagen seen in previous experiments were caused by its decreased intracellular accumulation (synthesis/protein expression). Nifedipine (50 μM) reduced ascorbate intracellular accumulation in cultured aortic smooth muscle cells compared to the control under water-soluble ascorbic acid as well as lipid soluble ascorbyl palmitate OR ANY OTHER FORM OF ASCORBATE THAT IS FAT SOLUBLE. It was observed that decreased collagen deposition by this channel blocker could be rescued by supplementation of AoSMC with increased levels of ascorbic acid or with ascorbyl palmitate.

These results confirm the adverse effects of channel blockers on collagen type I and collagen type IV ECM deposition. Ascorbate supplementation could reverse the inhibition of these critical ECM components necessary for maintaining optimal structural integrity of the arterial wall. Ascorbic acid is essential for the hydroxylation of lysine and proline, a precondition for optimum crosslinking of collagen and elastin and, thus, for maintaining the integrity of the vascular wall [9, 14]. As mentioned earlier, Ca²⁺ channel blockers such as verapamil, ditiazem and others were shown to significantly decrease collagen deposition in the ECM in human vascular smooth muscle cells and human dermal fibroblasts. These drugs inhibited the expression of fibrillary collagens type I and II and of basement membrane collagen type IV and increased the proteolytic activity of the 72-kDa type IV collagenase [8].

As in human scurvy, complete dietary ascorbate deprivation has been shown to cause marked alteration in the structural integrity of vascular connective tissue [10, 15, 16]. In a previous study, it was found that hyposcorbemic Gulo−/− mice developed early atherosclerosis accompanied by the deposition of Lp(a) in the intima and deeper layers of the vascular wall, while age-matched mice with high amounts of dietary ascorbate supplementation did not develop atherosclerosis and no Lp(a) detectable in the vascular wall [10]. Disruption of the endothelial layer, impairment of the basement membrane and the structural disintegration of the ECM paralleled the development of scurvy [15]. Since channel blockers have been shown to decrease ascorbate-dependent collagen I and IV, use of channel blockers can lead to increased probability of development of atherosclerosis.

Regular intake of ascorbic acid results in a variable absorption rate between 70 to 95%, with the degree of absorption decreasing as intake increases [17]. Fractional human absorption of ascorbic acid may be as low as 33% at high intake (1.25 g), but it can reach 98% at low intake (<200 mg) [17]. Ascorbate concentrations over renal re-absorption threshold pass freely into the urine and are excreted. At high dietary doses (corresponding to several hundred mg/day in humans) ascorbate is accumulated in the body until the plasma levels reach the renal resorption threshold, which is about 1.5 mg/dL in men and 1.3 mg/dL in women [18]. Ascorbate at concentrations in the plasma larger than this value (thought to represent body saturation) is rapidly excreted in the urine with a half-life of about 30 minutes. Concentrations less than this threshold value are actively retained by the kidneys, and the excretion half-life for the remainder of the vitamin C store in the body thus increases greatly, with the half-life lengthening as the body stores are depleted. This half-life rises until it is as long as 83 days by the onset of the first symptoms of scurvy [18].

Ascorbic acid is absorbed in the body by both active transport and simple diffusion. Sodium-dependent active transport—sodium-ascorbate co-transporters (SVCTs) and hexose transporters (GLUTs)—are the two transporters required for absorption. SVCT1 and SVCT2 import the reduced form of ascorbate across plasma membrane [19]. GLUT1 and GLUT3 are the two glucose transporters, and transfer only the dehydroascorbic acid form of ascorbic acid [20]. Although dehydroascorbic acid is absorbed at a higher rate than ascorbate, the amount of dehydroascorbic acid found in plasma and tissues under normal conditions is low, as cells rapidly reduce dehydroascorbic acid to ascorbate [21, 22]. Thus, SVCTs appear to be the predominant system for vitamin C transport in the body. Possible mechanism of ascorbate rescuing effect on channel blocker induced inhibition could be due to competitive inhibition of channel blockers. These conclusions are supported by the rescuing action of increased levels of ascorbate supplementation in channel blocker-dependent inhibition of collagen deposition. Kuo et al. [23] reported that hydrophobic 1,4-dihydropyridine compounds (nifedipine and nicardipine) inhibited Na⁺-dependent and Na⁺-independent (K⁺ substituting Na⁺) accumulation of ascorbic acid in human intestinal Caco-2 cells. Ebersole and Molinoff report that ascorbate inhibits binding of dihydropyridine calcium channel blockers [24]. A study by Grossmann et al. demonstrated that co-infusion of ascorbate and quinidine (K+ blocker) abolished ascorbate-dependent venodilation in human veins [25]. The instant disclosure shows that a method of counteracting an inhibition of collagen synthesis in a subject suffering from cardiovascular disease and taking prescribed amount of at least one of a calcium channel blocker, a sodium channel blocker and a combination thereof, and administering an effective amount of an ascorbate as an adjunct therapy to increase the collagen synthesis in an intracellular and extra cellular matrix of the subject. It is evident that adjunct therapy with ascorbate such as ascorbic acid and/or ascorbyl palmitate will not only increase collagen production and maintain the internal and external milieu of the subject cardiovascular health but also counter the negative effect of the channel blockers for the subject who is human.

The absence of inhibitory effects of channel blockers on ascorbyl palmitate dependent synthesis, ECM deposition of collagen and on accumulation of intracellular ascorbate from channel blockers apparently could be explained by different pathways of intracellular uptake independent from ion-channel-associated transporters, i.e. through hydrophobic portions of the cellular membrane.

It has been proposed that heart disease is an early form of scurvy, a condition that increases the need for biological repair of weakened arterial walls due to impaired collagen synthesis in the body. Ascorbic acid is essential for the hydroxylation of lysine and proline, a precondition for optimum crosslinking of collagen and elastin and, thus, for maintaining the integrity of the vascular wall. The major pathway of ascorbic acid cellular uptake is dependent on ion-channel dependent transporters.

Therefore, the objective in this study was to evaluate possible interference of select channel blockers on intracellular accumulation and cellular functions of ascorbate in relation to extracellular matrix (ECM) formation. Effects of select Na- and Ca-channel blockers on collagen synthesis and deposition were evaluated in cultured human dermal fibroblasts and aortic smooth muscle cells by immunoassay. Collagen type I and collagen type IV formation were decreased by the calcium and sodium channel blockers showing inhibition of collagen synthesis. The inhibitory effects of the calcium and sodium channel blockers on percent collagen synthesis was reversed by introducing ascorbic acid and ascorbyl palmitate in the cell culture. Sodium [Na] and calcium [Ca] channel blockers are used in the prevention, treatment and symptom management of various forms of cardiovascular diseases including hypertension, arrhythmia, coronary artery disease as well as other forms of occlusive cardiovascular disease. From this study, it can be concluded that calcium and sodium channel blockers have a direct inhibitory effect on collagen synthesis, which is mitigated by treatment with ascorbic acid and ascorbyl palmitate.

These studies confirm the adverse effects of channel blockers on collagen type I and type IV deposition, which are the key ECM components essential for maintaining optimal structural integrity of the arterial walls. Ascorbate supplementation reversed channel blocker inhibition of these collagen types synthesis and deposition. The results of this study imply the benefits of ascorbate and ascorbate palmitate supplementation in medical management of cardiovascular disease in order to compensate for adverse effects of channel blockers.

### REFERENCES

1—Sampson K J and Kass R S: Anti-arrhythmic drugs, in Goodman & Gillman The Pharmacological Basis of Therapeutics 12th Edition, ed Brunton L L, McGraw Medical, 2011.
2—National Library of Medicine: Sodium channel blockers. Updated 2011. Accessed May 26, 2016 http://www.nlm.nih.gov/cgi/mesh/2011/MB_cgi?mode=&term=Sodium+Channel+Blockers
3—Klabunde R E. Cardiovascular pharmacology concepts: Sodium-Channel Blockers (Class I Antiarrhythmics). Revised Dec. 1, 2011, accessed May 26, 2016. http://www.cvpharmacology.com/antiarrhy/sodium-blockers
4—Binshtok A M, Bean B P and Woolf C J. Inhibition of nociceptors by TRPV1-mediated entry of impermeant sodium channel blockers. Nature 2007; 449: 607-610.
5—Klabunde R E. Cardiovascular pharmacology concepts: Calcium-Channel Blockers (CCBs). Revised Mar. 22, 2015, accessed May 26, 2016. http://www.cvpharmacology.com/vasodilator/CCB
6—Psaty B M, Heckbert S R, Koepsell T D, Siscovick D S, Raghunathan T E, Weiss N S, Rosendaal F R, Lemaitre R N, Smith N L and Wahl P W. The risk of myocardial infarction associated with antihypertensive drug therapies. JAMA 1995; 274:620-625.
7—Furberg C D, Psaty B M and Meyer J V. Nifedipine: Dose-related increase in mortality in patients with coronary heart disease. Circulation 1995; 92:1326-1331.
8—Roth M, Eickelberg O, Köhler E, Erne P and Block L H. Ca²⁺ channel blockers modulate metabolism of collagens within the extracellular matrix. PNAS 1996; 93:5478-5482.
9—Rath M and Pauling L. Hypothesis: Lipoprotein(a) is a surrogate for ascorbate. PNAS 1990; 87: 6204-6207.
10—Cha J, Niedzwiecki A and Rath M. Hypoascorbemia induces atherosclerosis and vascular deposition of lipoprotein(a) in transgenic mice. Am J Cardiovasc Dis 2015; 5(1):53-62.
11—Ivanov V, Ivanova S, Roomi M W, Kalinovsky T, Niedzwiecki A and Rath M. Extracellular matrix-mediated control of aortic smooth muscle cell growth and migration by a combination of ascorbic acid, lysine, proline, and catechins. J Cardiovasc Pharmacol 2007; 50(5):541-7.
12—Ivanov V, Ivanova S, Kalinovsky T, Niedzwiecki A and Rath M. Plant-derived micronutrients suppress monocyte adhesion to cultured human aortic endothelial cell layer by modulating its extracellular matrix composition. J Cardiovasc Pharmacol 2008 52(1):55-65.
13—Lane D J R and Lawen A. Non-transferrin iron reduction and uptake are regulated by transmembrane ascorbate cycling in K562 cells. J Biol Chem 2008; 283(19): 12701-12708.
14—Pinnell S R. Regulation of collagen biosynthesis by ascorbic acid: a review. Yale J Biol Med 1985; 58: 553-9.
15—Gore I, Fujinami T and Shirahama T. Endothelial changes produced by ascorbic acid deficiency in guinea pigs. Arch Pathol 1965; 80: 371-6.
16—Maeda N, Hagihara H, Nakata Y, Hiller S, Wilder J and Reddick R. Aortic wall damage in mice unable to synthesize ascorbic acid. PNAS 2000; 97: 841-618.
17—Levine M, Conry-Cantilena C, Wang Y, Welch R W, Washko P W, Dhariwal K R, Park J B, Lazarev A, Graumlich J F, King J and Cantilena L R. Vitamin C pharmacokinetics in healthy volunteers: evidence for a recommended dietary allowance. PNAS 1996; 93 (8): 3704-9.
18—Oreopoulos D G, Lindeman R D, VanderJagt D J, Tzamaloukas A H, Bhagavan H N, Garry P J. Renal excretion of ascorbic acid: effect of age and sex. J Am Coll Nutr 1993; 12 (5): 537-42.
19—Savini I, Rossi A, Pierro C, Avigliano L and Catani MV. SVCT1 and SVCT2: key proteins for vitamin C uptake. Amino Acids 2008; 34 (3): 347-55.
20—Rumsey S C, Kwon O, Xu G W, Burant C F, Simpson I and Levine M. Glucose transporter isoforms GLUT1 and GLUT3 transport dehydroascorbic acid. J Biol Chem 1997; 272 (30): 18982-9.
21—May J M, Qu Z C, Neel D R and Li X. Recycling of vitamin C from its oxidized forms by human endothelial cells. Biochim Biophys Acta 2003; 1640 (2-3): 153-61.
22—Packer L. Vitamin C and redox cycling antioxidants. In Fuchs J, Packer L. Vitamin C in health and disease. 1997, New York: M. Dekker.
23—Kuo S M, Lin C P and Morehouse H F. Dihydropyridine calcium channel blockers inhibit ascorbic acid accumulation in human intestinal Caco-2 cells. Life Sci 2001; 68 (15): 1751-60.
24—Ebersole B and Molinoff P B. Identification of ascorbate as an endogenous substance that irreversibly inhibits binding of dihydropyridine calcium channel blockers. J Neurochem 1992; 58(4): 1300-7.
25—Grossmann M, Dobrev D, Himmel H M, Ravens U and Kirch W. Ascorbic acid-induced modulation of venous tone in humans. Hypertension 2001; 37:949-954.

What is claimed is:

1. A method of increasing an ascorbate dependent-ECM deposition of collagen type I and IV, comprising:
administering an ascorbate as an adjunct therapy to increase the ascorbate dependent-ECM deposition of collagen type I and IV synthesis in an intracellular milieu and extra cellular matrix of the subject having a disease and treated by a calcium channel blocker, a sodium channel blocker and a combination thereof.

2. The method of claim 1, wherein the calcium channel blocker, the sodium channel blocker and the combination thereof is at least one of the following:
a) Capsaicin, a natural sodium channel blocker;
b) Lidocaine, a sodium channel blocker;
c) Quinidine, a sodium channel blocker;
d) Ditiazem, a calcium channel blocker;
e) Nifedipine, a calcium channel blocker; and
f) Verapamil, a calcium channel blocker.

3. The method of claim 1, wherein the ascorbate is in form of ascorbic acid.

4. The method of claim 1, wherein the ascorbate is in form of calcium ascorbate, magnesium ascorbate, another form of mineral ascorbate or a combination thereof.

5. The method of claim 1, wherein the ascorbate is in form of an ascorbyl palmitate or another fat soluble form of ascorbate

6. The method of claim 1, wherein the ascorbate is any combination of the ascorbate forms of claims 3 to 5.

7. A method of countering an inhibition of collagen type I and IV synthesis in a subject suffering from a disease due to usage of a channel blocker, comprising:
administering an ascorbate to the subject as an adjunct treatment with the channel Mocker; wherein the channel blocker is at least one of a calcium channel blocker, the sodium channel blocker and the combination thereof;

8. A method according to claim 7 where the disease includes the prevention and/or treatment of hypertension.

9. A method according to claim 1 where the disease includes the prevention and/or treatment of arrhythmia.

10. A method according to claim 7 where the disease includes the prevention and/or treatment of coronary artery disease, cerebrovascular disease or any other form of occlusive vascular disease

11. A method according to claim 7 where the disease includes the prevention and/or treatment of angina pectoris.
